SEQUENCE LISTING (42 sequences)

SEQ ID NO: 1 - DNA, 4390 bp, Homo sapiens
  misc_feature (1252)..(1254): start codon
tatcctatccctatcctagcttttaacctgagccagagctcactacacaggttcctggctatcgagtctgaatctgcactactcaacttataaactgtctgcagacacctgttagggaaattgctgatcatgggcggcaggatctgaactcgctttaccttcttgtttggagcacagggaccgcccagctagaggagcaccagcgcactgcgccccagccctgggcgagggtgcggaggatttgttctcggtgcaatcctgctggcgcttttccggggttctgcgcggatccagctccccatctctgctcctacacacacaaaagaaaacaactctcgattggaagttgtggaattttctcagcccctacgaggcgcggggattctccagccccggccctcctcccgccagcctgaggtctccttcgctcgcctgccttgctagggaccgcagtccctcagccgcagctgggtctgtccgccccgcctttgccctcgccttttcccggggcggatttggtgaagtcggcctcaagtccaggaggtctgtcttcgccgggccagctctcgcggaactggggggtagagagcaaagggagagattcgtggaagggaagggaggtaggggtggcgcaaacgcccagagtatcaaacttgggggtggcacagtaggtgacagcagcagctgcaggtggtggctggggacccgcgagggggcgcccctctgggtagggtctggctgagcgggcttgcaagcccgggaggcggctgagagaccctggacactgttcctgctccctcgccaccaaaacttctcctccagtcccctcccctgcaggaccatcgcccgcagcctctgcacctgttttcttgtgtttaagggtggggtttgcccccctccccacgctcccatctctgatcctcccaccttcacccgcccaccccgcgagtgagtgcggtgcccaggcgcgcttggcctgagaggtcggcagcagacccggcagcgccaaccgcccagccgctctgactgctccggctgcccgcccgcgcggcgcgggctgtcctggaccctaggaggggacggaaccggacttgcctttgggcaccttccagggccctctccaggtcggctggctaatcatcggacagacggactgcacacatcttgtttccgcgtctccgcaaaaacgcgaggtccaggtcagttgtagactcttgtgctggttgcaggccaagtggacctgtactgaaaatgggtccaataggtgcagaggctgatgagaaccagacagtggaagaaatgaaggtggaacaatacgggccacaaacaactcctagaggtgaactggtccctgaccctgagccagagcttatagatagtaccaagctgattgaggtacaagttgttctcatattggcctactgctccatcatcttgcttggggtaattggcaactccttggtgatccatgtggtgatcaaattcaagagcatgcgcacagtaaccaactttttcattgccaatctggctgtggcagatcttttggtgaacactctgtgtctaccgttcactcttacctataccttaatgggggagtggaaaatgggtcctgtcctgtgccacctggtgccctatgcccagggcctggcagtacaagtatccacaatcaccttgacagtaattgccctggaccggcacaggtgcatcgtctaccacctagagagcaagatctccaagcgaatcagcttcctgattattggcttggcctggggcatcagtgccctgctggcaagtcccctggccatcttccgggagtattcgctgattgagatcatcccggactttgagattgtggcctgtactgaaaagtggcctggcgaggagaagagcatctatggcactgtctatagtctttcttccttgttgatcttgtatgttttgcctctgggcattatatcattttcctacactcgcatttggagtaaattgaagaaccatgtcagtcctggagctgcaaatgaccactaccatcagcgaaggcaaaaaaccaccaaaatgctggtgtgtgtggtggtggtgtttgcggtcagctggctgcctctccatgccttccagcttgccgttgacattgacagccaggtcctggacctgaaggagtacaaactcatcttcacagtgttccacatcatcgccatgtgctccacttttgccaatccccttctctatggctggatgaacagcaactacagaaaggctttcctctcggccttccgctgtgagcagcggttggatgccattcactctgaggtgtccgtgacattcaaggctaaaaagaacctggaggtcagaaagaacagtggccccaatgactctttcacagaggctaccaatgtctaaggaagctgtggtgtgaaaatgtatggatgaattctgaccagagctatgaatctggttgatggcggctcacaagtgaaaactgatttcccattttaaagaagaagtggatctaaatggaagcatctgctgtttaattcctggaaaactggctgggcagagcctgtgtgaaaatactggaattcaaagataaggcaacaaaatggtttacttaacagttggttgggtagtaggttgcattatgagtaaaagcagagagaagtacttttgattattttcctggagtgaagaaaacttgaacaagaaattggtattatcaaagcattgctgagagacggtgggaaaataagttgactttcaaatcacgttaggacctggattgaggaggtgtgcagttcgctgctccctgcttggcttatgaaaacaccactgaacagaaatttctccagggagccacaggctctccttcatcgcattttgatttttttgttcattctctagacaaaatccatcagggaatgctgcaggaaacgattgccaactatacgaatggcttcgaggagataaactgaaatttgctatataattaatattttggcagatgataggggaactcctcaacactcagtgggccaattgttcttaaaaccaattgcacgtttggtgaaagtttcttcaactctgaatcaaaagctgaaattctcagaattacaggaaatgcaaaccatcatttaatttctaatttcaagttacatccgctttatggagatactatttagataacaagaatacaacttgatacttttattgttatacctttttgaacatgtatgatttctgttgttatttacctttttaaacagataaatatttttttttcattttagagtagcggaatctaatcttaatctaatcttttaggagtatatttcagagaaattccaagcacaccagtatgaccatccttatttcagaaatgacaatgcatagaggaaaagtaatatgtgcaaagcctccgaagaggatggttaagtaaagacttaggttaccagtatcaggctttcgtttttgtatgtaggtagctctactgcctcctcttaaaaccaacaaaggaaagagagactggctgcaaacttttagaaggaatggcttcgaatagggttcctgggaggaatcccgaggaaatagacgctgctgctctgctgattgtctccactatcctgttttgctcctacccactaatccagcctgggaggctctgggcattagcggaaggcttcaccacaaggagacaggagcgagtattccataggcatgcgctcctagtggcacgagtggcttgggtcaggatcaaagagtgaaggattcggaagtcagctatctggagagagagagagattgtgttttattcgtgtcccatagctttcctatcctatccctatcctagcttttaacctgagccagagctcactacacaggttcctggctatcgagtctgaatctgcactactcaacttataaactgtctgcagacacctgttagggaaattgctgatcatgggcggcaggatctgaactcgctttaccttcttgtttggagcacagggaccgcccagctagaggagcaccagcgcactgcgccccagccctgggcgagggtgcggaggatttgttctcggtgcaatcctgctggcgcttttccggggttctgcgcggatccagctccccatctctgctcctacacacacaaaagaaaacaactctcgattggaagttgtggaattttctcagcccctacgaggcgcggggattctccagccccggccctcctcccgccagcctgaggtctccttcgctcgcctgccttgctagggaccgcagtccctcagccgcagctgggtctgtccgccccgcctttgccctcgccttttcccggggcggatttggtgaagtcggcctcaagtccaggaggtctgtcttcgccgggccagctctc

SEQ ID NO: 2 - DNA, 20 bp, Homo sapiens
cctctgcacctattggaccc

SEQ ID NO: 3 - DNA, 21 bp, Homo sapiens
gtttgtggcccgtattgttcc

SEQ ID NO: 4 - DNA, 20 bp, Homo sapiens
ggccactgttctttctgacc

SEQ ID NO: 5 - DNA, 19 bp, Homo sapiens
gttgacattgacagccagg

SEQ ID NO: 6 - DNA, 1147 bp, Rattus sp.
atgggcccattaggtgcagaggcagatgagaatcaaactgtagaagtgaaagtggaactctatgggtcggggcccaccactcctagaggtgagttgccccctgatccagagccggagctcatagacagcaccaaactggttgaggtgcaggtggtccttatactggcctattgttccatcatcttgctgggcgtagttggcaactctctggtaatccatgtggtgatcaaattcaagagcatgcgcacagtaaccaacttttttattgccaacctggctgtggcggatcttttggtgaacaccctgtgcctgccattcactcttacctataccttgatgggggagtggaaaatgggtccagttttgtgccatttggtgccctatgcccagggtctggcagtacaagtgtccacaataactttgacagtcattgctttggaccgacatcgttgcattgtctaccacctggagagcaagatctccaagcaaatcagcttcctgattattggcctggcgtggggtgtcagcgctctgctggcaagtccccttgccatcttccgggagtactcactgattgagattattcctgactttgagattgtagcctgtactgagaaatggcccggggaggagaagagtgtgtacggtacagtctacagcctttccaccctgctaatcctctacgttttgcctctgggcatcatatctttctcctacacccggatctggagtaagctaaagaaccacgttagtcctggagctgcaagtgaccattaccatcagcgaaggcacaaaacgaccaaaatgctcgtgtgcgtggtagtggtgtttgcagtcagctggctgcccctccatgccttccaacttgctgtggacatcgacagccatgtcctggacctgaaggagtacaaactcatcttcaccgtgttccacattattgcgatgtgctccaccttcgccaacccccttctctatggctggatgaacagcaactacagaaaagctttcctctcagccttccgctgtgagcagaggttggatgccattcactcggaggtgtccatgaccttcaaggctaaaaagaacctggaagtcaaaaagaacaatggcctcactgactctttttcagaggccaccaacgtgtaag

SEQ ID NO: 7 - DNA, 20 bp, Homo sapiens
ctgcacctattggacccatt

SEQ ID NO: 8 - DNA, 20 bp, Homo sapiens
ctctgcacctattggaccca

SEQ ID NO: 9 - DNA, 20 bp, Homo sapiens
gcctctgcacctattggacc

SEQ ID NO: 10 - DNA, 20 bp, Homo sapiens
cagcctctgcacctattgga

SEQ ID NO: 11 - DNA, 20 bp, Homo sapiens
cgtattgttccaccttcatt

SEQ ID NO: 12 - DNA, 20 bp, Homo sapiens
ccgtattgttccaccttcat

SEQ ID NO: 13 - DNA, 20 bp, Homo sapiens
cccgtattgttccaccttca

SEQ ID NO: 14 - DNA, 20 bp, Homo sapiens
gcccgtattgttccaccttc

SEQ ID NO: 15 - DNA, 20 bp, Homo sapiens
ggcccgtattgttccacctt

SEQ ID NO: 16 - DNA, 20 bp, Homo sapiens
ttttccactcccccattaag

SEQ ID NO: 17 - DNA, 20 bp, Homo sapiens
attttccactcccccattaa

SEQ ID NO: 18 - DNA, 20 bp, Homo sapiens
cattttccactcccccatta

SEQ ID NO: 19 - DNA, 20 bp, Homo sapiens
ccattttccactcccccatt

SEQ ID NO: 20 - DNA, 20 bp, Homo sapiens
cccattttccactcccccat

SEQ ID NO: 21 - DNA, 20 bp, Homo sapiens
ctcaatcagcgaatactccc

SEQ ID NO: 22 - DNA, 20 bp, Homo sapiens
gatctcaatcagcgaatact

SEQ ID NO: 23 - DNA, 20 bp, Homo sapiens
gccacaatctcaaagtccgg

SEQ ID NO: 24 - DNA, 20 bp, Homo sapiens
ggccacaatctcaaagtccg

SEQ ID NO: 25 - DNA, 20 bp, Homo sapiens
gcattttggtggttttttgc

SEQ ID NO: 26 - DNA, 20 bp, Homo sapiens
ccagcattttggtggttttt

SEQ ID NO: 27 - DNA, 20 bp, Homo sapiens
ccacacacaccagcattttg

SEQ ID NO: 28 - DNA, 20 bp, Homo sapiens
ccaccaccacacacaccagc

SEQ ID NO: 29 - DNA, 20 bp, Homo sapiens
cgcaaacaccaccaccacac

SEQ ID NO: 30 - DNA, 20 bp, Homo sapiens
gccagctgaccgcaaacacc

SEQ ID NO: 31 - DNA, 20 bp, Homo sapiens
gcctttctgtagttgctgtt

SEQ ID NO: 32 - DNA, 20 bp, Homo sapiens
ggaaagcctttctgtagttg

SEQ ID NO: 33 - DNA, 20 bp, Homo sapiens
ggccgagaggaaagcctttc

SEQ ID NO: 34 - DNA, 20 bp, Homo sapiens
ccactgttctttctgacctc

SEQ ID NO: 35 - DNA, 20 bp, Homo sapiens
gccactgttctttctgacct

SEQ ID NO: 36 - DNA, 20 bp, Homo sapiens
gggccactgttctttctgac

SEQ ID NO: 37 - DNA, 20 bp, Homo sapiens
ggggccactgttctttctga

SEQ ID NO: 38 - DNA, 20 bp, Rattus sp.
cctctgcacctaatgggccc

SEQ ID NO: 39 - DNA, 20 bp, Rattus sp.
ccatggtaatccgccgctcc

SEQ ID NO: 40 - DNA, 20 bp, Homo sapiens
cccaggttatccacgtctcc

SEQ ID NO: 41 - DNA, 21 bp, Homo sapiens
gcctcggcttgtcacatctgc

SEQ ID NO: 42 - DNA, 3747 bp, Homo sapiens
  misc_feature (496)..(498): start codon
gaattcggccgctgagagaccctggacactgttcctgctccctcgccaccaaaacttctcctccagtcccctcccctgcaggaccatcgcccgcagcctctgcacctgttttcttgtgtttaagggtggggtttgcccccctccccacgctcccatctctgatcctcccaccttcacccgcccaccccgcgagtgagtgcggtgcccaggcgcgcttggcctgagaggtcggcagcagacccggcagcgccaaccgcccagccgctctgactgctccggctgcccgcccgcgcggcgcgggctgtcctggaccctaggaggggacggaaccggacttgcctttgggcaccttccagggccctctccaggtcggctggctaatcatcggacagacggactgcacacatcttgtttccgcgtctccgcaaaaacgcgaggtccaggtcagttgtagactcttgtgctggttgcaggccaagtggacctgtactgaaaatgggtccaataggtgcagaggctgatgagaaccagacagtggaagaaatgaaggtggaacaatacgggccacaaacaactcctagaggtgaactggtccctgaccctgagccagagcttatagatagtaccaagctgattgaggtacaagttgttctcatattggcctactgctccatcatcttgcttggggtaattggcaactccttggtgatccatgtggtgatcaaattcaagagcatgcgcacagtaaccaactttttcattgccaatctggctgtggcagatcttttggtgaacactctgtgtctaccgttcactcttacctataccttaatgggggagtggaaaatgggtcctgtcctgtgccacctggtgccctatgcccagggcctggcagtacaagtatccacaatcaccttgacagtaattgccctggaccggcacaggtgcatcgtctaccacctagagagcaagatctccaagcgaatcagcttcctgattattggcttggcctggggcatcagtgccctgctggcaagtcccctggccatcttccgggagtattcgctgattgagatcattccggactttgagattgtggcctgtactgaaaagtggcctggcgaggagaagagcatctatggcactgtctatagtctttcttccttgttgatcttgtatgttttgcctctgggcattatatcattttcctacactcgcatttggagtaaattgaagaaccatgtcagtcctggagctgcaaatgaccactaccatcagcgaaggcaaaaaaccaccaaaatgctggtgtgtgtggtggtggtgtttgcggtcagctggctgcctctccatgccttccagcttgccgttgacattgacagccaggtcctggacctgaaggagtacaaactcatcttcacagtgttccacattatcgccatgtgctccacttttgccaatccccttctctatggctggatgaacagcaactacagaaaggctttcctctcggccttccgctgtgagcagcggttggatgccattcactctgaggtgtccgtgacattcaaggctaaaaagaacctggaggtcagaaagaacagtggccccaatgactctttcacagaggctaccaatgtctaaggaagctgtggtgtgaaaatgtatggatgaattctgaccagagctatgaatctggttgatggcggctcacaagtgaaaactgatttcccattttaaagaagaagtggatctaaatggaagcatctgctgtttaattcctggaaaactggctgggcagagcctgtgtgaaaatactggaattcaaagataaggcaacaaaatggtttacttaacagttggttgggtagtaggttgcattatgagtaaaagcagagagaagtacttttgattattttcctggagtgaagaaaacttgaacaagaaattggtattatcaaagcattgctgagagacggtgggaaaataagttgactttcaaatcacgttaggacctggattgaggaggtgtgcagttcgctgctccctgcttggcttatgaaaacaccactgaacagaaatttctccagggagccacaggctctccttcatcgcattttgatttttttgttcattctctagacaaaatccatcagggaatgctgcaggaaacgattgccaactatacgaatggcttcgaggagataaactgaaatttgctatataattaatattttggcagatgataggggaactcctcaacactcagtgggccaattgttcttaaaaccaattgcacgtttggtgaaagtttcttcaactctgaatcaaaagctgaaattctcagaattacaggaaatgcaaaccatcatttaatttctaatttcaagttacatccgctttatggagatactatttagataacaagaatacaacttgatacttttattgttatacctttttgaacatgtatgatttctgttgttattcctattggagctaagtttgtctacactaaaatttaaatcagactagagaataatttttgtggcatgttgtaacatttcacagtatttacaagctatttttgcacaggtacatagctctcatgtatttaaagaacactgcagtgttattttctttgaaattcatcctccacggacccattcatactaaataaaacaatgtaattacattaaaatggacctatctgtaagaggtactaaaaacactggattcatttcatcttgcaaatgttgtatttcaaaccagtttcacataagttatttgtcttcttttcaaaataattagctatatttttatataatatgaatatatacataaaaattgtttctataaattgtagaacatagatgctacagtattttttatttaattatattatgaataaaattgttatttcaatagtacccaaccaaagatgcttaaaaaccttctatgttcataaaaaataacaactgagatgttaaaatagtcatacgtctttagatgctattaaagtttcattagtcatatttttgtaaatatgacagaatttgtgaatatatttttaaagcaaaaaacttcaacatgcatatgatatatagttacaacattaattttatgaactggagagctttactttgtggatatatttaaaattcatattatagctcctattaaattccttccatgatagatataaaggactggtttttaagtgcactgcacttctggaatactgaaaaagaatgaaaacaatatgttagattaggtgtaagactttaagaagcgaacaaaaagtaatgtatatctgtaatatataatcaaatgattcatttttctgttagactaggcaaattgttcaaaaataacctttttgtcttttaagtagcagtcactttgcttaagatgctaatagaaaactgtggttaaagatttaccctccctcttggtgaattattacactgtaagaaatgtatatgctactgtgttacatgttgtattagtaaattattagaatccaattaatgattcaattaacatatatcttatccaattcattatgtcaattcattaataaaataccttttatgtagaggctttatgttgcaattaaaaagttgggaaaatgaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

(19) United States
(12) Patent Application Publication
(10) Pub. No.: US 2009/0162349 A1
(43) Pub. Date: Jun. 25, 2009

(54) METHOD FOR PREVENTION OR TREATMENT OF DISEASES OR DISORDERS RELATED TO EXCESSIVE FORMATION OF VASCULAR TISSUE OR BLOOD VESSELS

(75) Inventors: Markku KOULU, Turku (FI); Jukka TUOHIMAA, Turku (FI); Ullamari PESONEN, Turku (FI); Jaana KALLIO, Turku (FI); Matti KARVONEN, Turku (FI)

Correspondence Address: ROTHWELL, FIGG, ERNST & MANBECK, P.C., 1425 K STREET, N.W., SUITE 800, WASHINGTON, DC, 20005, US

(73) Assignee: PHASE ONE PHARMA OY LTD., Turku (FI)

(21) Appl. No.: 12/388,894

(22) Filed: Feb. 19, 2009

Related U.S. Application Data
(63) Continuation of application No. 11/372,212, filed on Mar. 10, 2006.
Application No. 11/372,212 is a division of application No. 10/462,039, filed on Jun. 16, 2003, now abandoned.
(60) Provisional application No. 60/509,044, filed on Jun. 27, 2002.

(51) Int. Cl. A61K 39/395; A61K 31/7088; A61K 38/00

(52) U.S. Cl. 424/130.1; 514/44; 514/2

(57) ABSTRACT

This invention concerns a method for treating or preventing a disease or disorder related to excessive formation of vascular tissue or blood vessels in a patient, said method comprising administering to said patient an agent affecting the NPY Y2 receptor.

### CROSS-REFERENCE TO RELATED APPLICATION

The present application is a continuation of U.S. patent application Ser. No. 11/372,212 filed on 10 Mar. 2006, which in turn is a divisional of U.S. patent application Ser. No. 10/462,039 filed on 16 Jun. 2003, which in turn is related to and claims priority under 35 U.S.C. § 119(e) to U.S. provisional patent application Ser. No. 60/509,044 filed 27 Jun. 2002, each incorporated herein by reference.

### FIELD OF THE INVENTION

This invention relates to methods for prevention or treatment of diseases or disorders related to excessive formation of vascular tissue or blood vessels, i.e. any disease or disorder in which angiogenesis is involved. The method is based on the use of targeted inhibition (or blocking) of neuropeptide Y (NPY) Y2 receptor mediated actions. The invention also concerns novel antisense oligonucleotides and their use in said methods as well as novel antisense oligonucleotides and their use in investigating the development of said diseases or disorders in experimental animals.

### BACKGROUND OF THE INVENTION

The publications and other materials used herein to illuminate the background of the invention, and in particular, cases to provide additional details respecting the practice, are incorporated by reference.

NPY is a neurotransmitter of the sympathetic nervous system, co-stored with noradrenaline in peripheral sympathetic nerve endings and released in response to strenuous sympathetic stimulation (Lundberg, Fried, et al. 1986 (1)). When released from peripheral nerve terminals to arterial periadventitia NPY causes direct endothelium-independent vasoconstriction via stimulation vascular smooth-muscle cell receptors (Edvinsson, Emson, et al. 1983 (2); Edvinsson 1985 (3); Abounader, Villemure, et al. 1995 (4)).

NPY is widely expressed in the central and peripheral nervous systems and has many physiological functions such as in the control of metabolism and endocrine functions and in regulation of cardiovascular homeostasis.

In addition to release from peripheral nerve endings to arterial periadventitia, NPY and NPY mRNA are also expressed extraneuronally in the endothelium of peripheral vessels (Loesch, Maynard, et al. 1992 (5); Zukowska-Grojec, Karwatowska-Prokopczuk, et al. 1998 (6)). The minor proportion of circulating NPY level, derived from the endothelial cells has been implicated to act as an autocrine and paracrine mediator and to stimulate its receptors Y1 and Y2 found on the endothelium (Sanabria and Silva 1994 (7); Jackerott and Larsson 1997 (8); Zukowska-Grojec, Karwatowska-Prokopczuk, et al. 1998 (6). In addition to NPY, the endothelium can also produce NPY[3-36], a more specific Y2 agonist, from circulating native NPY by a serine protease dipeptidyl peptidase IV (Mentlein, Dahms, et al. 1993 (9)). Recent studies have demonstrated that stimulation of endothelial NPY receptors leads to vasodilatation (Kobari, Fukuuchi, et al. 1993 (10); Torffvit & Edvinsson 1997 (11)) primarily through Y2 receptor activation (You, Edvinsson, et al. 2001 (12)). In experimental study settings NPY has shown mitogenic action on smooth muscle tissue and vascular growth promoting properties. Grant and Zukowska demonstrated that NPY is a potent angiogenic factor that has promising potential to the revascularization of ischemic tissue (Grant and Zukowska 2000 (13)). The mitogenic effect of NPY has been speculated to be mediated via Y1 or Y2 receptors (Zukowska-Grojec, Pruszczyk et al. 1993 (14); Nilsson and Edvinsson 2000 (15)) and vascular growth promotion is mediated by inducible Y1, Y2, or Y5 receptors (Zukowska-Grojec Z, Karwatowska-Prokopczuk et al. 1998 (6)).

Angiogenesis is involved in a variety of human diseases. The NPY system and Y2 receptor has been shown to play a role in the regulation of the formation of blood vessels and to be active during the development of retinopathy (Zukowska-Grojec Z, et. al. 1998 (6); Lee E W, et al. 2003 (16); Ekstrand A J et al. 2003 (17)). Thus, identification of agents blocking the NPY mediated action thorough Y2 receptor may have potential applications in the treatment of a variety of human diseases.

It was recently reported that a rather common Leu7Pro polymorphism located in the signal peptide of the prepro-NPY is associated with higher prevalence of diabetic retinopathy in type 2 diabetic patients (Niskanen, Voutilainen-Kaunisto et al. 2000 (18)). This study linked the NPY system with the development of diabetic retinopathy. However, it has not earlier been suggested to treat or prevent such diseases by affecting the NPY Y2 receptor.

### SUMMARY OF THE INVENTION

According to one aspect, this invention concerns a method for treating or preventing a disease or disorder related to excessive formation of vascular tissue or blood vessels in a patient, said method comprising administering to said patient an agent affecting the NPY Y2 receptor.

According to another aspect, this invention concerns an antisense oligonucleotide having a length ranging from 7 to 40 nucleotides, wherein said antisense oligonucleotide is complementary to any sequence of the human NPY Y2 receptor mRNA.

According to a third aspect, the invention concerns an antisense oligonucleotide having a length ranging from 7 to 40 nucleotides, wherein said antisense oligonucleotide is complementary to any sequence of animal NPY Y2 receptor mRNA.

According to a fourth aspect, the invention concerns a method for investigating the development of a disease or disorder related to excessive formation of vascular tissue or blood vessels in an experimental animal using an antisense oligonucleotide having a length ranging from 7 to 40 nucleotides, wherein said antisense oligonucleotide is complementary to any sequence of animal NPY Y2 receptor mRNA.

According to a fifth aspect, the invention concerns a pharmaceutical composition comprising a therapeutically effective amount of an antisense oligonucleotide or a mixture of antisense oligonucleotides in a pharmaceutically acceptable carrier, said oligonucleotide having a length ranging from 7 to 40 nucleotides and being complementary to any sequence of the human NPY Y2 receptor mRNA.

According to a sixth aspect, the invention concerns an expression vector including a nucleotide sequence encoding an antisense oligonucleotide having a length ranging from 7 to 40 nucleotides and being complementary to any sequence of the human or animal NPY Y2 receptor mRNA, in a manner which allows expression of said antisense oligonucleotide in a mammalian cell.

### BRIEF DESCRIPTION OF THE DRAWINGS

FIGS. 1A and 1B show the human neuropeptide Y2 receptor mRNA (Genbank Accession No. NM_{—}000910), illustrated as cDNA (SEQ ID NO:1). Three examples of antisense oligonucleotides are inserted in bold letters: AS-1 (SEQ ID NO:2), AS-2 (SEQ ID NO:3) and AS-3 (SEQ ID NO:4). Also a published PCR primer (SEQ ID NO:5) complementary to the human neuropeptide Y2 receptor mRNA is inserted.

FIG. 2 shows the protein coding region of the rat neuropeptide Y2 receptor mRNA, illustrated as cDNA (SEQ ID NO:6). Nucleotide number 1 represents the start codon.

FIG. 3 shows the development of induced retinopathy in rat puppies treated by i) vehicle, ii) scramble oligonucleotide ((SEQ ID NO: 39) containing 20 thioate modified bases), or iii) an antisense oligonucleotide ((SEQ ID NO: 38), containing 20 thioate modified bases) complementary to NPY Y2 receptor mRNA

FIGS. 4A-4D show the efficacy of studied antisense molecules and their combinations in the prevention of tubular structures by hTERT-HUVEC cells. (AS-1, namely 5′-CCTCTGCACCTATTGGACCC-3′, (SEQ ID NO:2); AS-2, namely 5′-GTTTGTGGCCCGTATTGTTCC-3′, (SEQ ID NO:3); AS-3, namely 5′-GGCCACTGTTCTTTCTGACC-3′, (SEQ ID NO:4); AS-1 control, sequence: 5′-CCCAGGTTATCCACGTCTCC-3′ (SEQ ID NO:40), and human vascular endothelial growth factor antisense (VEGF-AS, sequence: 5′-GCCTCGGCTTGTCACATCTGC-3, (SEQ ID NO:41)).

FIG. 5 shows as photographs the efficacy of different single antisense molecules and their combinations in the prevention of endothelial cell tube formation by hTERT-HUVEC cells. (AS-1, namely 5′-CCTCTGCACCTATTGGACCC-3′, (SEQ ID NO: 2); AS-2, namely 5′-GTTTGTGGCCCGTATTGTTCC-3′, (SEQ ID NO: 3); AS-3, namely 5′-GGCCACTGTTCTTTCTGACC-3′, (SEQ ID NO: 4); AS-1 control, sequence: 5′-CCCAGGTTATCCACGTCTCC-3′ (SEQ ID NO: 40).

### DETAILED DESCRIPTION OF THE INVENTION

Our current results conducted using living cells derived from humans demonstrate that the antisense molecules directed against human NPY Y2 receptor mRNA are effective inhibitors of angiogenesis. Thus any compound preventing the NPY Y2 receptor transmission could be a potent inhibitor of tumor angiogenesis, and could have a more general interest in every disease in which angiogenesis is involved.

The wording “disease or disorder related to excessive formation of vascular tissue or blood vessels in a patient” shall be understood to cover any such disease or disorder which can be treated or prevented by an agent to antagonize or block or prevent or modify the action of the NPY Y2 receptor.

Examples of diseases, the treatment of which could be clinically greatly benefited from the down regulation, or blockage of Y2 receptor, or prevention of the action of naive NPY or fragments of NPY (e.g. NPY 3/36 or 13-16, which are endogenous) on Y2 receptor are non-neoplastic pathologic conditions characterized by excessive angiogenesis, such as neovascular glaucoma, any form of retinopathy, all proliferative retinopathies including proliferative diabetic retinopathy, retinopathy of prematurity, macular degeneration, maculopathy, micro- or macrovascular eye complications caused by diabetes, nephropathy, diabetic nephropathy, rubeosis iridis, hemangiomas, angiofibromas, and psoriasis. This method is also effective for treating subjects with tumors and neoplasms, including malignant tumors and neoplasms, such as blastomas, carcinomas or sarcomas, and especially highly vascular tumors and neoplasms. Some examples of tumors that can be treated with the invention include epidermoid tumors, squamous tumors, such as head and neck tumors, colorectal tumors, prostate tumors, breast tumors, lung tumors, including small cell and nonsmall cell lung tumors, pancreatic tumors, thyroid tumors, ovarian tumors, and liver tumors, vascularized skin cancers, including squamous cell carcinoma, basal cell carcinoma, and skin cancers that can be treated by suppressing the growth of neovasculature. Other cancers that can be treated by the method according to this invention include Kaposi's sarcoma, CNS neoplasms (neuroblastomas, capillary hemangioblastomas, meningiomas and cerebral metastases), melanoma, gastrointestinal and renal carcinomas and sarcomas, rhabdomyosarcoma, glioblastoma, preferably glioblastoma multiforme, and leiomyosarcoma.

However, the diseases or disorders are not restricted to the aforementioned list. Furthermore, the wording “disease or disorder related to excessive formation of vascular tissue or blood vessels in a patient” includes further prevention of diseases or disorder directly derivable from the aforementioned conditions. Thus, for example, this wording also includes the prevention of predisposition to vision loss and blindness, which are consequences of retinopathy. Also metabolic diseases and cardiovascular diseases are included.

The diseases or disorders to be prevented or treated according to the method of this invention are particularly retinopathies or retinal neovascularization processes in diabetes like type I or type II diabetes, other metabolic diseases or cardiovascular diseases.

The term “NPY Y2 receptor” shall be understood to mean a receptor encoded by NPY Y2 receptor gene and mRNA (Gehlert, Beavers et al. 1996 (19); Rose P M, Fernandes et al. 1995 (20)) or active for NPY or a peptide fragment of NPY. Such a fragment can, for example, be the peptide fragment of NPY₃₋₃₆, NPY₁₃₋₃₆ (Wimalawansa 1995 (21), Grandt et al. 1996 (22)) or N-acetyl [Leu(28,31)] NPY 24-36 (Smith-White and Potter 1999 (23)) or the like.

The term “agent” shall be understood to include the compound itself (racemic form as well as isomers), and any pharmaceutically acceptable derivatives thereof, such as salts or esters and templates. It shall be also understood to include peptide compounds and derivatives antagonising NPY Y2 receptor. It shall be also understood to include agents that direct the action of endogenous NPY Y2 receptor agonists and ligands away from NPY Y2 receptor, thus attenuating NPY Y2 receptor action. It shall be also understood to include any agent aimed at influencing any phases of NPY Y2 receptor transcription and translation processes, and any device or instrument (genetic or other) needed for this mentioned action.

The active agent to be administered can in principle be either an NPY Y2 antagonist, or a combination of an antagonist in a said NPY Y2 receptor and an agonist or an antagonist in another receptor, for example in NPY Y5 receptor. The same agent can thus be an antagonist in said NPY Y2 receptor and an agonist or an antagonist in another receptor. The same agent can thus be also a partial agonist.

According to a preferable embodiment of this invention, the agent is an NPY receptor antagonist. Y2 receptor antagonists have been described before in the literature. As an example can be mentioned BIIE 0246 (Doods, Gaida et al 1998 (24)). The suitable agent is, however, not restricted to the aforementioned examples. Any compound acting as a Y2 receptor antagonist is useful in the method according to this invention.

It is also believed that an agent blocking or influencing/inhibiting the action of dipeptidyl peptidase IV and therefore prevention of the catabolism of NPY to NPY₃₋₃₆ and the action of NPY₃₋₃₆ and native NPY towards NPY Y2 receptor could be useful. As an example can be mentioned Dipeptidyl Peptidase IV Inhibitor P32/98 (Pospisilik, Stafford et al. 2002 (25)) and dipeptidyl peptidase IV inhibitor isoleucine thiazolidide (Rahfeld J, Schierhorn et al 1991 (26)). The suitable agent is, however, not restricted to the aforementioned examples. Alternatively, an antisense oligonucleotide, an aptamer or an antibody directed to dipeptidyl peptidase IV would also be useful.

It is also believed that a combination of action on the Y1 and Y5 receptor in addition to Y2 antagonism and could be useful.

An Y2-receptor antagonistic molecule with a property of intrinsic NPY receptor stimulating activity on Y1- and or Y5-receptors, which by acting on NPY Y2 and/or Y1 and/or Y5-receptors prevents the development and progression of retinopathy and nephropathy, and which blocks inappropriate (excessive) vasculoproliferative actions (potential retinopathy and nephropathy and related conditions promoting effects of excess endogenous NPY) of endogenous NPY and growth hormone and insulin like growth factor-I. Thus it is also believed that antagonising NPY Y2 action prevents the development and progression of retinopathy and nephropathy through reducing growth hormone and insulin like growth factor-I.

Thus, according to another embodiment of this invention the Y2 receptor antagonist is also a Y1 or/and Y5-receptor agonist or antagonist.

According to a further embodiment, a separate Y1 and/or Y5 receptor agonist or antagonist is administered in combination with the Y2 receptor agonist.

According to further embodiments, this invention also concerns any method by which the prevention or down regulation of the action of NPY Y2 receptor is possible such a the use of an antisense oligonucleotide, modified nucleotide, sequence of combination of different kinds of nucleotides or any other sequence able to antagonize the action of NPY Y2 receptor or prevent or modify the NPY Y2 receptor synthesis, modification, activity, ligand binding, metabolism or degradation. The antisense oligonucleotide can be a DNA molecule or an RNA molecule. Ribozymes cleaving the NPY Y2 receptor mRNA are also included.

The ribozyme technology is described for example in the following publications: Ribozyme protocols: Turner, Philip C (editor) (27); Rossi J J. 1999 (28); and Ellington A D, Robertson M P, Bull J. 1997 (29).

Also small interfering RNA molecules would be useful (30).

According to a further alternative, the agent affecting the NPY Y2 receptor can be an antibody raised against said receptor or raised against an Y2-specific epitope on the NPY peptide. NPY receptor specific antibodies are known in the art, but they have been used only to study the distribution of the Y2-receptor and other NPY receptors.

According to still another alternative, the agent affecting the NPY Y2 receptor can be an aptamer affecting the Y2 receptor or a Y2-specific NPY-conformation. An aptamer is an oligonucleotide affecting the protein. Many antisense oligonucleotides have also the ability to interact with peptides. There are known NPY aptamers affecting the Y2-specific NPY-conformation and thereby preventing the NPY from binding to the Y2 receptor. Also aptamers affecting the NPY receptor are known. For publications relating to aptamers, see references 31-33.

The novel antisense oligonucleotides complementary to any sequence of the human or animal NPY Y2 receptor mRNA, which according to the broadest definition can be of a length ranging from 7 to 40 nucleotides, have preferably a length ranging from 15 to 25 nucleotides, most preferably about 20 nucleotides.

The term “complementary” means that the antisense oligonucleotide sequence can form hydrogen bonds with the target mRNA sequence by Watson-Crick or other base-pair interactions. The term shall be understood to cover also sequences which are not 100% complementary. It is believed that lower complementarity, even as low as 50% or more, may work. However, 100% complementarity is preferred.

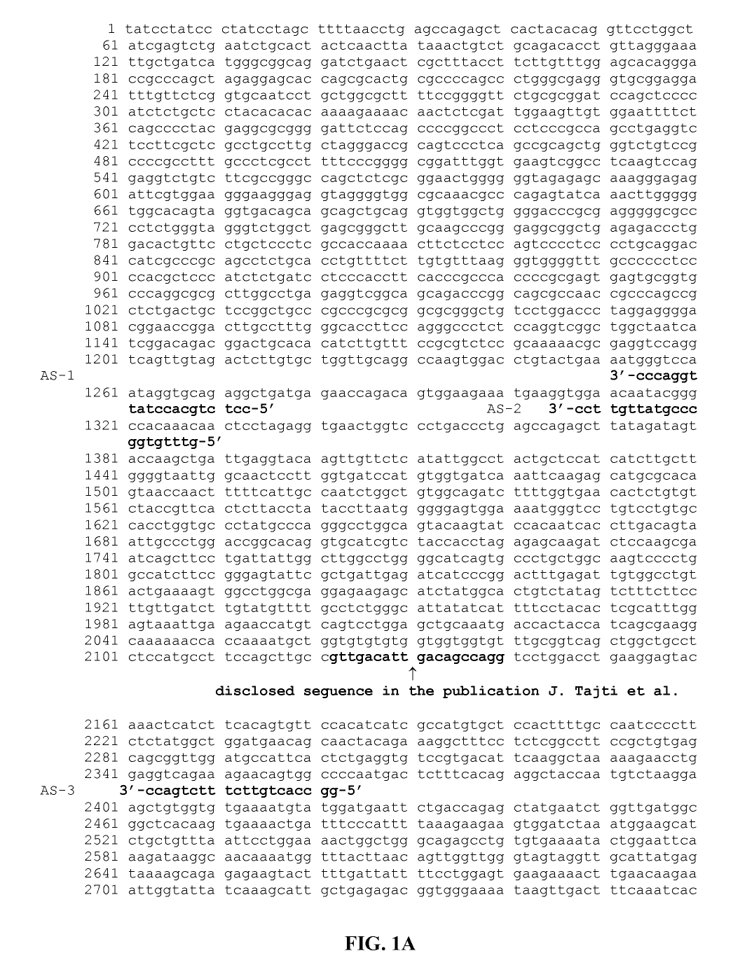

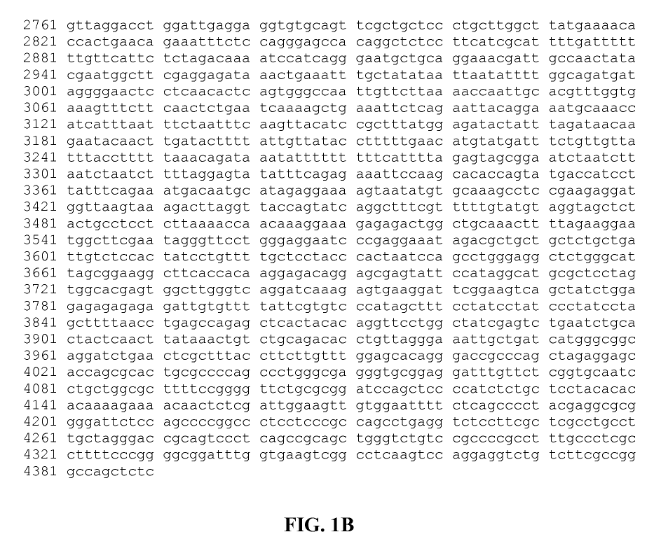

In FIGS. 1A and 1B disclosing the human NPY Y2 receptor mRNA (shown as cDNA; SEQ ID NO: 1), three preferable antisense oligonucleotides of 20-21 nt are inserted in bold letters. Although a suitable antisense oligonucleotide could be created to any string of 7 to 40 nucleotides in the shown mRNA comprising 4390 nucleotides, it is believed that the best target region in the mRNA is found in the beginning of the mRNA sequence, especially in the regions 1 nt to 2100 nt and 2200 nt to 2500 nt of SEQ ID NO:1, more preferably the regions 1200 nt to 2100 nt and 2200 nt to 2400 nt of SEQ ID NO:1, and most preferable the target regions defined by the specific antisense oligonucleotides shown herein. Furthermore, regions with inter se binding nucleotides (hairpins etc.) should be avoided. The publication J Tajti et al., 1999 (34) discloses a PCR primer, namely 5′-CTGGCTGTCAATGTCAAC-3′ (SEQ ID NO:5), which is complementary to the human NPY Y2 receptor mRNA (shown as cDNA) as indicated in FIGS. 1A and 1B. This sequence was not, however, disclosed as a useful antisense. A revised sequence for human NPY Y2 receptor mRNA is available in Genbank and is set forth in SEQ ID NO:42. The coding region of SEQ ID NO: 1 and SEQ ID NO:42 are identical, except for a C at nucleotide 2187 of SEQ ID NO:1 and a T at corresponding nucleotide 1431 of SEQ ID NO:42. The antisense oligonucleotides disclosed herein are identical in both sequences.

Normal, unmodified antisense oligonucleotides have low stability under physiological conditions because of its degradation by enzymes present in the living cell. It is therefore highly desirable to modify the antisense oligonucleotide according to known methods so as to enhance its stability against chemical and enzymatic degradation.

Modifications of antisense oligonucleotides are extensively disclosed in prior art. Reference is made to Draper et al., U.S. Pat. No. 5,612,215, which in turn lists a number of patents and scientific papers concerning this technique. It is known that removal or replacement of the 2′-OH group from the ribose unit gives a better stability. Eckstein et al., WO 92/07065 and U.S. Pat. No. 5,672,695 discloses the replacement of the ribose 2′-OH group with halo, amino, azido or sulfhydryl groups. Sproat et al., U.S. Pat. No. 5,334,711, discloses the replacement of hydrogen in the 2′-OH group by alkyl or alkenyl, preferably methyl or allyl groups. Furthermore, the internucleotidic phosphodiester linkage can, for example, be modified so that one or more oxygen is replaced by sulfur, amino, alkyl or alkoxy groups. Preferable modification in the internucleotide linkages are phosphorothioate linkages. Also the base in the nucleotides can be modified. Usman and Blatt, 2000 (35), disclose a new class of nuclease-resistant ribozymes, where the 3′ end of the antisense oligonucleotide is protected by the addition of an inverted 3′-3′ deoxyabasic sugar.

A preferable antisense oligonucleotide is a nucleotide chain wherein one or more of the internucleotide linkages are modified, and/or wherein the oligonucleotide contains locked nucleic acid (LNA) modifications and/or wherein the oligonucleotide contains peptide nucleic acid (PNA) modifications. Margaret F Taylor, 2001 (36) discloses a great variety of modifications. According to this publication, the sugar unit can, for example also be replaced by a morpholino group. This publication further discloses that different kinds of modifications inhibits the mRNA translation in different ways. All kinds of modifications described in this article are incorporated herein by reference.

The PNA technology is described in Ray A and Norden, 2000 (37).

Another preferable antisense oligonucleotide is a nucleotide chain wherein one or more of the sugar units are modified, and/or one or more of the internucleotide linkages are modified, and/or one or more of the bases are modified and/or the oligonucleotide is end-protected by an inverted deoxyabasic sugar.

As an example of preferred embodiments can be mentioned any NPY Y2 receptor targeted sequence of antisense deoxynucleotide phosphorothioates or oligonucleotides containing locked nucleic acids or peptide nucleic acids or ribozyme. Specific preferable examples are AS-1, which is 5′-CCT CTG CAC CTA TTG GAC CC-3′ (SEQ ID NO:2); AS-2, which is 5′-GTTTGTGGCCCGTATTGTTCC-3′, (SEQ ID NO:3) and AS-3, which is 5′-GGCCACTGTTCTTTCTGACC-3′, (SEQ ID NO:4) or longer sequences comprising these chains of nucleotides. All antisense sequences that can recognize and bind any part of the human NPY Y2 receptor mRNA sequence, including all occurring variations due to polymorphism in the human NPY Y2 receptor gene are also concerned.

As further examples of useful antisenses can be mentioned the sequences listed below (SEQ ID NO:7 to SEQ ID NO:37):

| | |
|---|---|
| 5′- CTGCACCTATTGGACCCATT -3′ | (SEQ ID NO:7) |
| 5′- CTCTGCACCTATTGGACCCA -3′ | (SEQ ID NO:8) |
| 5′- GCCTCTGCACCTATTGGACC -3′ | (SEQ ID NO:9) |
| 5′- CAGCCTCTGCACCTATTGGA -3′ | (SEQ ID NO:10) |
| 5′- CGTATTGTTCCACCTTCATT -3′ | (SEQ ID NO:11) |
| 5′- CCGTATTGTTCCACCTTCAT -3′ | (SEQ ID NO:12) |
| 5′- CCCGTATTGTTCCACCTTCA -3′ | (SEQ ID NO:13) |
| 5′- GCCCGTATTGTTCCACCTTC -3′ | (SEQ ID NO:14) |
| 5′- GGCCCGTATTGTTCCACCTT -3′ | (SEQ ID NO:15) |
| 5′- TTTTCCACTCCCCCATTAAG -3′ | (SEQ ID NO:16) |
| 5′- ATTTTCCACTCCCCCATTAA -3′ | (SEQ ID NO:17) |
| 5′- CATTTTCCACTCCCCCATTA -3′ | (SEQ ID NO:18) |
| 5′- CCATTTTCCACTCCCCCATT -3′ | (SEQ ID NO:19) |
| 5′- CCCATTTTCCACTCCCCCAT -3′ | (SEQ ID NO:20) |
| 5′- CTCAATCAGCGAATACTCCC -3′ | (SEQ ID NO:21) |
| 5′- GATCTCAATCAGCGAATACT -3′ | (SEQ ID NO:22) |
| 5′- GCCACAATCTCAAAGTCCGG -3′ | (SEQ ID NO:23) |
| 5′- GGCCACAATCTCAAAGTCCG -3′ | (SEQ ID NO:24) |
| 5′- GCATTTTGGTGGTTTTTTGC -3′ | (SEQ ID NO:25) |
| 5′- CCAGCATTTTGGTGGTTTTT -3′ | (SEQ ID NO:26) |
| 5′- CCACACACACCAGCATTTTG -3′ | (SEQ ID NO:27) |
| 5′- CCACCACCACACACACCAGC -3′ | (SEQ ID NO:28) |
| 5′- CGCAAACACCACCACCACAC -3′ | (SEQ ID NO:29) |
| 5′- GCCAGCTGACCGCAAACACC -3′ | (SEQ ID NO:30) |
| 5′- GCCTTTCTGTAGTTGCTGTT -3′ | (SEQ ID NO:31) |
| 5′- GGAAAGCCTTTCTGTAGTTG -3′ | (SEQ ID NO:32) |
| 5′- GGCCGAGAGGAAAGCCTTTC -3′ | (SEQ ID NO:33) |
| 5′- CCACTGTTCTTTCTGACCTC -3′ | (SEQ ID NO:34) |
| 5′- GCCACTGTTCTTTCTGACCT -3′ | (SEQ ID NO:35) |
| 5′- GGGCCACTGTTCTTTCTGAC -3′ | (SEQ ID NO:36) |
| 5′- GGGGCCACTGTTCTTTCTGA -3′ | (SEQ ID NO:37) |

Combinations of antisenses are also useful. Two or more of the antisense sequences SEQ ID NOs: 2-4 or SEQ ID NOs: 7-37 can be used, or any of these sequences can be used in combination with other antisense oligonucleotides such as human vascular endothelial growth factor antisense (VEGF-AS, 5′-GCCTCGGCTTGTCACATCTGC-3′, (SEQ ID NO:41).

The suitable agent is, however, not restricted to the aforementioned examples. Any compound acting as a Y2 receptor antagonist or attenuating Y2 receptor action is useful in the method according to this invention.

According to a further embodiment, this invention also concerns a novel antisense oligonucleotide having a length ranging from 7 to 40 nucleotides, wherein said antisense oligonucleotide is complementary to any sequence of animal NPY Y2 receptor mRNA. The experimental animal is preferable a rodent such as a rat or mouse. The term “complementary” shall have the same meaning as presented above for the human sequence.

These antisense oligonucleotides preferably contains one or more modifications as described above.

The invention concerns methods for investigating the development of a disease or disorder related to excessive formation of vascular tissue or blood vessels, particularly any form of retinopathy, in an experimental animal using such antisense oligonucleotides complementary to animal NPY Y2 receptor mRNA.

As an example can be mentioned any NPY Y2 receptor targeted sequence of antisense deoxynucleotide phosphorothioates or oligonucleotides containing locked nucleic acids or peptide nucleic acids or ribozyme. As an example of the sequence is a sequence containing 5′-CCT CTG CAC CTA ATG GGC CC-3′ (SEQ ID NO:38) corresponding to rat NPY Y2 mRNA. The suitable agent is, however, not restricted to the aforementioned example.

For the purpose of this invention, the NPY receptor active agent can be administered by various routes. The suitable administration forms include, for example, oral or topical formulations; parenteral injections including intraocular, intravitreous, intravenous, intramuscular, intraperitoneal, intradermal and subcutaneous injections; and transdermal, intraurethral or rectal formulations; and inhaled and nasal formulations. Suitable oral formulations include e.g. conventional or slow-release tablets and gelatine capsules.

The antisense oligonucleotides according to this invention can be administered to the individual by various methods. According to one method, the sequence may be administered as such, as complexed with a cationic lipid, packed in a liposome, incorporated in cyclodextrins, bioresorbable polymers or other suitable carrier for slow release administration, biodegradable nanoparticle or a hydrogel. For some indications, antisense oligonucleotides may be directly delivered ex vivo to cells or tissues with or without the aforementioned vehicles.

In addition to direct delivery of the antisense oligonucleotide, an antisense oligonucleotide-encoding sequence can be incorporated into an expression vector, and said vector administered to the patient. The expression vector can be a DNA sequence, such as a DNA plasmid capable of eukaryotic expression, or a viral vector. Such a viral vector is preferably based on an adenovirus, an alphavirus, an adeno-associated virus, a retrovirus or a herpes virus. Preferably, the vector is delivered to the patient in similar manner as the antisense oligonucleotide described above. The delivery of the expression vector can be systemic, such as intravenous, intramuscular or intraperitoneal administration, or local delivery to target tissue.

The required dosage of the NPY receptor active agents will vary with the particular condition being treated, the severity of the condition, the duration of the treatment, the administration route and the specific compound being employed.

The invention will be illuminated by the following non-restrictive Experimental Section.

### Experimental Section

The present study was undertaken to determine the impact of NPY Y2 receptor targeted intervention on neovascularization and development of retinopathy. Development of retinopathy was induced to newborn rats by cyclic hyperoxia and following relative ischemia-induced retinal neovascularization. Hyperoxemia is toxic to developing retinal vessels causing damage and hypoxia in the retina. After moving to normal air, relative hypoxia follows further promoting neovascularization of the retina.

Three groups of rat puppies were subjected for different treatments; 1) vehicle, 2) NPY Y2 receptor targeted antisense oligonucleotide sequence, and 3) scramble oligonucleotide sequence containing the same oligonucleotides as NPY Y2 receptor targeted antisense oligonucleotide sequence. The treatments were administered intraperitoneally. The retinal vessels were investigated and retinopathic changes were compared between treatment groups.

Retinopathy was assessed after injection of fluorescent-labelled dextran to the circulation. The eyes were flat-mounted on slides and the retinal vessels were visualized and investigated by fluorescence microscopy. Statistical differences were calculated between the study groups.

### Retinal Neovascularization Protocol

Study protocol was approved by the Joint Ethics Committee of Turku University. Development of retinopathy was induced to newborn rats (Sprague Dawley) by cyclic hyperoxia and following relative ischemia. Hyperoxia is toxic to developing retinal vessels causing damage and hypoxia in the retina, which induces neovascularization. After moving to normal air, relative hypoxia follows further promoting neovascularization of the retina. Hypoxia is one of the major causes of retinal neovascularization in human retinopathies also. The newborn rats were kept in a hyperoxic incubator with their mothers. Retinal neovascularization was induced simultaneously for all three groups of puppies. One treatment group consisted originally of 7 puppies, which underwent cyclic hyperoxia at the age of 3 days, continued until at the age of 14 days and remained in normal room air from the age of 14 to 17 days. The amount of oxygen inside the incubator was kept at 40% and 80% in 12 hour cycles for 10 days (days from 3 to 13).

### Treatments

The three groups of puppies were subjected for different treatments; 1) plain vehicle, 2) NPY Y2 receptor targeted antisense oligodeoxynucleotide sequence (5′-CCT CTG CAC CTA ATG GGC CC-3′ (SEQ ID NO:38), containing 20 thioate modified bases) diluted in vehicle and 3) scramble oligodeoxynucleotide sequence containing the same deoxynucleotides as NPY Y2 receptor targeted antisense oligodeoxynucleotide sequence but in a random order (5′-CCA TGG TAA TCC GCC GCT CC-3′ (SEQ ID NO:39), containing 20 thioate modified bases) diluted in vehicle. The treatments were administered intraperitoneally. The retinal vessels were investigated and retinopathic changes were compared between treatment groups. The used NPY Y2 receptor targeted antisense deoxynucleotide sequence was designed complementary to next 20 bases from NPY Y2 gene transcription initiation codon (ATG).

### Assessment of Retinopathy and Retinal Neovascularization

At the age of 20 days, rats were decapitated and eyes were collected. Retinopathy and retinal neovascularization was assessed after an injection of fluorescent-labelled dextran to the circulation trough heart puncture. One eye from each puppy was used for visualization of retinal vessels. The eyes were flat-mounted on slides and the retinal vessels were visualized and investigated by fluorescence microscopy. Pictures of retinas were acquired using a Leica DMR/DC 100 microscope and Leica DC Wiever software.

### Statistical Methods

The amount of retinal capillaries was analyzed by counting the amount of vessels crossed by a constant length line using plot profile analysis (Image-J 2.6 program). Each retina was analyzed in 3-5 representative areas and the mean values were used for further statistical analysis. Only unfolded retinal preparations were used in order to avoid artificial images of neovascularization. Five eyes from study group 1, and four eyes from study groups 2 and 3 were found unfolded and used for fluorescence microscopy and statistical analyses. Differences between study populations were calculated using Oneway anova followed by post hoc tests (Tukey HSD). P-value less than 0.05 was considered statistically significant. The results are expressed as mean±SD and range.

### Results

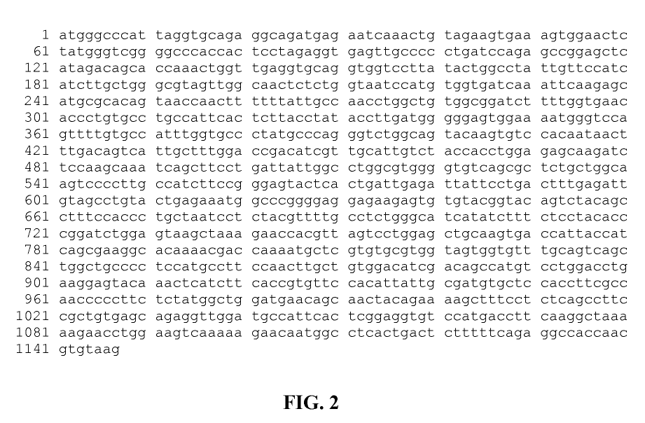

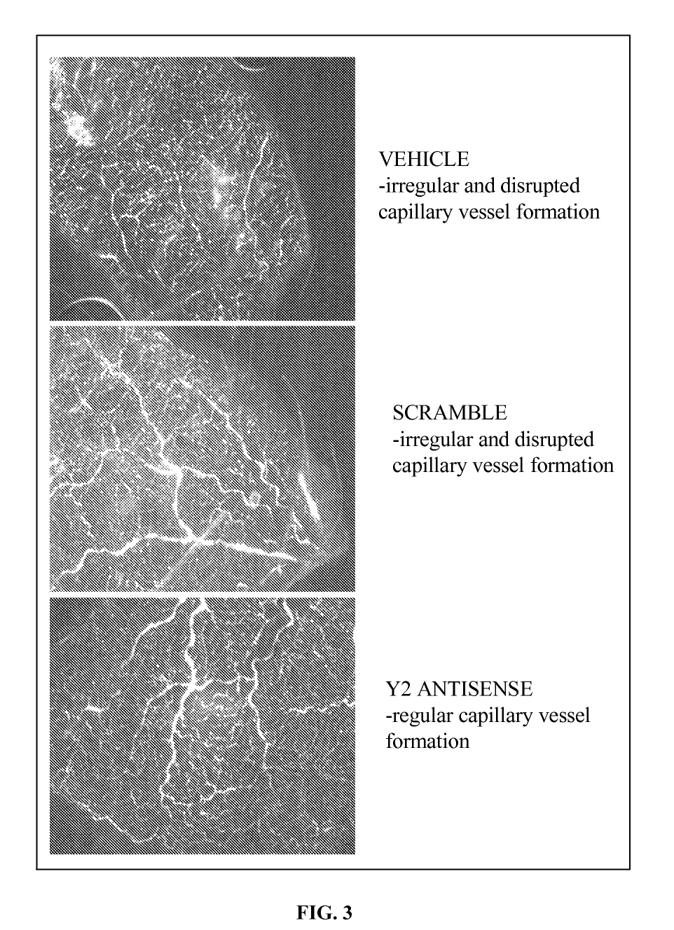

Retinal neovascularization and retinopathy was statistically significantly different between the treatment groups (p<0.001, Oneway anova). In vehicle and scramble treatment groups, the fluorescein images showed clearly an irregular and disrupted retinal capillary vessel formation, which was accompanied with blurred fluorescent emitting areas (FIG. 3). In Y2-antisense treatment group capillary vessel formation was regular and continuous and gives an impression of healthy retina without observable pathological changes. In post hoc analyses the Y2-antisense treatment group had statistically significantly less neovascularization, when compared to both vehicle treatment group (p<0.001 mean difference 5.40, 95% confidence interval for the difference 2.48-8.33), and to scramble treatment group (p<0.001 mean difference 6.53, 95% confidence interval for the difference 3.76-9.31). There was no difference in retinal neovascularization between vehicle and scramble treatment groups.

Table 1 below shows the mean values of quantitated neovascularization, representing retinopathy, in the three different study groups. The development of retinopathy was evident in vehicle and scramble treated groups of puppies, whereas prevented in NPY Y2 antisense treated group.

TABLE 1
Characteristics and Statistical Analysis of The
Retinal Preparations of Different Treatment Groups.

| | | | p-value for statistical |
|---|---|---|---|
| Treatment group, n | Mean ± SD | Range | significance |
| Vehicle, 4 | 29.99 ± 2.40 | 28.20-33.30 | |
| Y2-antisense, 4 | 24.58 ± 0.84 | 23.75-25.75 | *<0.001 #<0.001 |
| Scramble, 5 | 31.12 ± 0.93 | 30.33-32.25 | *0.527 |

*Tukey HSD, compared to Vehicle.
#Tukey HSD, compared to Scramble.

This study demonstrates that development of retinopathy and retinal neovascularizations can be prevented by NPY Y2-receptor targeted oligonucleotide antisense therapy, evidenced by comparison to plain vehicle and control non Y2-antisense deoxyoligonucleotide sequence. The result of this study first time emphasizes the role of NPY Y2-receptor in the treatment and prevention of retinopathy and retinal neovascularization.

Our finding of prevention of retinopathy and inappropriate vascular proliferation with NPY Y2 receptor targeted antisense therapy is novel. Only one previous study has linked NPY-system and potentially altered NPY action with diabetic retinopathy (Niskanen, Voutilainen-Kaunisto et al. 2000 (18)). This finding is of therapeutic potential for prevention and treatment of diabetic retinopathy and closely related diseases due to inappropriate vascular proliferation. Therefore diabetic nephropathy is also potentially preventable and treatable with NPY Y2 receptor targeted therapy, since diabetic nephropathy is also associated with in appropriate vessel growth and vascular tissue mitogenesis (Del Prete, Anglani et al. 1998 (38)). In addition, elevated immunoreactive NPY concentrations has been associated with diabetic nephropathy (Satoh, Satoh et al. 1999 (39)).

Hypoxia induce vascular proliferation is commonly used experimental model for studying the mechanisms involved in pathophysiology of retinopathy and effects of novel therapies to treat and prevent retinopathy (Smith, Shen et al. 1999 (40); Smith, Kopchick et al. 1997 (41); Ozaki, Seo et al. 2000 (42)). The used retinopathy model has its limitations but can be considered sufficient and useful in order to elucidate receptor level mechanisms leading to and involved in the patophysiology of variety of retinopathies, since vascular damage and ischemia are essentially involved in the development of retinal neovascularization in all retinopathies. Preventing NPY Y2 receptor action blocks retinal neovascularization and is therefore an excellent target for treatment of diabetes associated retinopathy, other proliferative retinopathies like retinopathy of prematurity and other ischemic retinopathies.

A further experiment was carried out in order to study the effect of single antisense molecules and their combinations in the prevention of endothelial cell tube formation by immortal human umbilical vein endothelial cells (hTERT-HUVECs).

### Cell Culture

Immortal human umbilical vein endothelial cells (hTERT-HUVECs) were obtained from Geron Corporation (Menlo Park, Calif., U.S.A.). hTERT-HUVECs were maintained on a gelatin-coated 100-mm dishes (Corning Costar, N.Y., U.S.A) in growth medium, composed of M199 medium (Gibco, Paisley, Scotland) supplement with 15% (v/v) heat-inactivated fetal bovine serum (Gibco BRL), 2 mM L-glutamine (Gibco BRL), 100 units/ml penicillin/streptomycin (Gibco BRL), 10 units/ml heparin (Sigma) and 20 μg/ml endothelial cell growth factor (Roche Biomolecules) at 37° C. in a humified incubator with 5% CO₂ atmosphere. Experiments were performed with cells between passages 20 and 24.

### Oligonucleotides

The following phosphorothioate oligonucleotides were synthesized: human neuropeptide Y2-receptor mRNA antisense molecules (AS-1, namely 5′-CCTCTGCACCTATTGGACCC-3′, (SEQ ID NO:2); AS-2, namely 5′-GTTTGTGGCCCGTATTGTTCC-3′, (SEQ ID NO:3); AS-3, namely 5′-GGCCACTGTTCTTTCTGACC-3′, (SEQ ID NO:4); AS-1 control, sequence: 5′-CCCAGGTTATCCACGTCTCC-3′ (SEQ ID NO:40), and human vascular endothelial growth factor antisense (VEGF-AS, sequence: 5′-GCCTCGGCTTGTCACATCTGC-3′, (SEQ ID NO:41)).

### Liposomes

N-(1-(2,3-dioleoyloxy)propyl)-N,N,N-trimethyl ammonium methylsulfate (DOTAP) and 1,2-dioleoyl-3-phosphatidylethanolamine (DOPE) were purchased from Avanti Polar Lipids. Cationic liposomes composed of DOTAP/DOPE (1:1 by mol) were prepared as previously described (Ruponen et al., 2001 (43)).

### Transfection Protocol

hTERT-HUVECs (5×10⁴ cells/well) were seeded onto gelatin-coated 48-multiwell plates (Corning Costar, N.Y., U.S.A) and incubated overnight. For transfection, the growth medium was replaced with 400 μl of transfection medium (M199 medium supplement with 2 mM L-glutamine and 100 units/ml penicillin/streptomycin). Oligonucleotides (final concentration 1 μM) and DOTAP/DOPE liposomes in sterile water were first diluted in MES-HEPES buffered saline (50 mM MES, 50 mM HEPES, 75 mM NaCl, pH 7.2) and then mixed together at a charge ratio +1. The transfection mixture was allowed to stand at room temperature for 20 min and the oligonucleotide/liposome complexes (100 μl) were added dropwise to each well.

### Endothelial Tube Formation Assay

After transfection for 4 h hTERT-HUVECs were harvested after trypsin treatment, suspended in growth medium (200 μl) and seeded in growth factor-reduced Matrigel (BD Biosciences) coated 96-well plates (Corning Costar, N.Y., U.S.A). After incubation for 3 h cells were fixed in 4% paraformaldehyde. The formation of tubular structures in each well (7 fields/well) was digitally captured using a Nicon Eclipse TE300 Inverted Microscope (Nicon, Tokyo, Japan) equipped with a Nicon F-601 digital camera (Nicon, Tokyo, Japan). Photographs were taken at 4× magnification.

The efficacy in prevention of formation of tubular structures by hTERT-HUVECs of all 5 synthesized antisense molecules were compared against each others alone and in combination. The number of tubular structures was analyzed by using Adobe Photoshop 5.5 (Adobe Systems Inc., San Jose, Calif., U.S.A) and the results were expressed as means±SEM of three independent experiments. A set of three experiments was repeated.

### Results

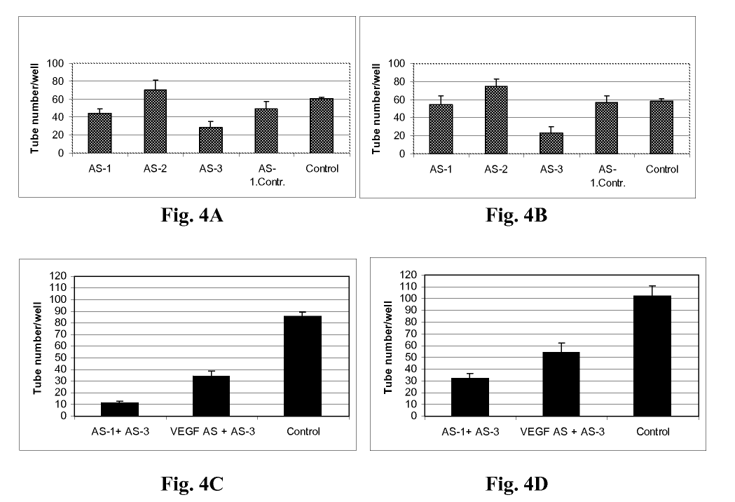

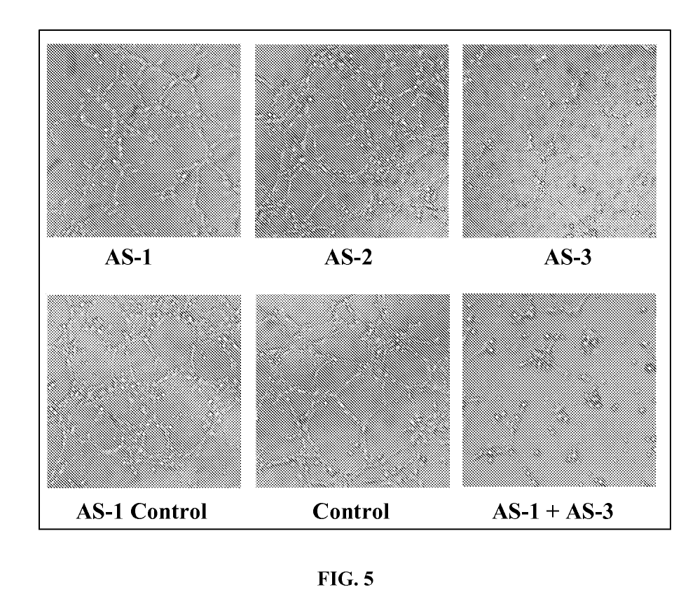

FIGS. 4A-4D demonstrate the efficacy of studied antisense molecules in the prevention of tubular structures by hTERT-HUVECs. FIGS. 4A and 4B represent repeated sets of three identical assays, and FIGS. 4c and 4d represent repeated set of other three identical assays. AS-3 antisense molecule shows the best efficacy in prevention of tubular structures formation by hTERT-HUVECs. AS-1 combined with AS-3 is the most potent alternative. The respective mean±SEM tube number/well values for single nucleotide assay 4A were: AS-1, 44.0±5.6; AS-2, 70.3±11.3; AS-3, 28±7.1; AS-1 control, 49.3±8.2; and control (non-treated), 60±1.8. For assay 4b: AS-1, 54.3±10.1; AS-2, 75.0±7.5; AS-3, 23.0±6.7; AS-1 control, 57.0±7.0; and control (non-treated), 58.0±2.9. The respective mean±SEM tube number/well values for combination nucleotide assays 4C was: AS-1±AS-3, 11.3±1.2; VEGF-AS±AS-3, 34.3±4.5; and control (non-treated), 85.7±3.4. For assay 4d: AS-1±AS-3, 32.3±4.3; VEGF-AS+AS-3, 54.0±8.0; and control (non-treated), 102.0±8.9.

It will be appreciated that the methods of the present invention can be incorporated in the form of a variety of embodiments, only a few of which are disclosed herein. It will be apparent for the expert skilled in the field that other embodiments exist and do not depart from the spirit of the invention. Thus, the described embodiments are illustrative and should not be construed as restrictive.

### BIBLIOGRAPHY

1. Lundberg J M, Terenius L, Hokfelt T et al. Neuropeptide Y (NPY)-like immunoreactivity in peripheral noradrenergic neurons and effects of NPY on sympathetic function. Acta Physiol Scand 1982; 116:477-80.
2. Edvinsson L, Emson P, McCulloch J, Tatemoto K, Uddman R. Neuropeptide Y: cerebrovascular innervation and vasomotor effects in the cat. Neurosci Lett 1983; 43:79-84.
3. Edvinsson L. Characterization of the contractile effect of neuropeptide Y in feline cerebral arteries. Acta Physiol Scand 1985; 125:33-41.
4. Abounader R, Villemure J G, Hamel E. Characterization of neuropeptide Y (NPY) receptors in human cerebral arteries with selective agonists and the new Y1 antagonist BIBP 3226. Br J Pharmacol 1995; 116:2245-50.
5. Loesch A, Maynard K I, Burnstock G. Calcitonin gene-related pep. Neuroscience 1992; 48:723-6.
6. Zukowska-Grojec Z, Karwatowska-Prokopczuk E, Rose W et al. Neuropeptide Y: a novel angiogenic factor from the sympathetic nerves and endothelium. Circ Res 1998; 83:187-95.
7. Sanabria P, Silva W I. Specific 1251 neuropeptide Y binding to intact cultured bovine adrenal medulla capillary endothelial cells. Microcirculation 1994; 1:267-73.
8. Jackerott M, Larsson L I. Immunocytochemical localization of the NPY/PYY Y1 receptor in enteric neurons, endothelial cells, and endocrine-like cells of the rat intestinal tract. J Histochem Cytochem 1997; 45:1643-50.
9. Mentlein R, Dahms P, Grandt D, Kruger R. Proteolytic processing of neuropeptide Y and peptide YY by dipeptidyl peptidase IV. Regul Pept 1993; 49:133-44.
10. Kobari M, Fukuuchi Y, Tomita M et al. Transient cerebral vasodilatory effect of neuropeptide Y mediated by nitric oxide. Brain Res Bull 1993; 31:443-8.
11. Torffvit O, Edvinsson L. Blockade of nitric oxide decreases the renal vasodilatory effect of neuropeptide Y in the insulin-treated diabetic rat. Pflugers Arch 1997; 434:445-50.
12. You J, Edvinsson L, Bryan R M, Jr. Neuropeptide Y-mediated constriction and dilation in rat middle cerebral arteries. J Cereb Blood Flow Metab 2001; 21:77-84.
13. Grant D S, Zukowska Z. Revascularization of ischemic tissues with SIKVAV and neuropeptide Y (NPY). Adv Exp Med Biol 2000; 476:139-54.
14. Zukowska-Grojec Z, Pruszczyk P, Colton C, Yao J, Shen G H, Myers A K, Wahlestedt C. Mitogenic effect of neuropeptide Y in rat vascular smooth muscle cells. Peptides 1993; 14(2):263-8.
15. Nilsson T, Edvinsson L. Neuropeptide Y stimulates DNA synthesis in human vascular smooth muscle cells through neuropeptide Y Y1 receptors. Can J Physiol Pharmacol 2000 March; 78(3):256-9.
16. Lee E W, Grant D S, Movafagh S, Zukowska Z. Impaired angiogenesis in neuropeptide Y (NPY)-Y2 receptor knockout mice. Peptides. 2003 January; 24(1):99-106.
17. Ekstrand A J, Cao R, Bjorndahl M, Nystrom S, Jonsson-Rylander A C, Hassani H, Hallberg B, Nordlander M, Cao Y. Deletion of neuropeptide Y (NPY) 2 receptor in mice results in blockage of NPY-induced angiogenesis and delayed wound healing. Proc Natl Acad Sci U S A. 2003 May 13; 100(10):6033-8.
18. Niskanen L, Voutilainen-Kaunisto R, Terasvirta M, Karvonen M K, Valve R, Pesonen U, Laakso M, Uusitupa M I, Koulu M. Leucine 7 to proline 7 polymorphism in the neuropeptide y gene is associated with retinopathy in type 2 diabetes. Exp Clin Endocrinol Diabetes 2000; 108(3):235-6.
19. Gehlert D R, Beavers L S, Johnson D, Gackenheimer S L, Schober D A, Gadski R A. Expression cloning of a human brain neuropeptide Y Y2 receptor. Mol Pharmacol 1996 February; 49(2):224-8.
20. Rose P M, Fernandes P, Lynch J S, Frazier S T, Fisher S M, Kodukula K, Kienzle B, Seethala R. Cloning and functional expression of a cDNA encoding a human type 2 neuropeptide Y receptor. J Biol Chem 1995 September 29; 270(39):22661-4.
21. Wimalawansa S J. Purification and biochemical characterization of neuropeptide Y2 receptor. J Biol Chem 1995; 270(31):18523-30.
22. Grandt D, Schimiczek M, Rascher W, Feth F, Shively J, Lee T D, Davis M T, Reeve J R Jr, Michel M C. Neuropeptide Y 3-36 is an endogenous ligand selective for Y2 receptors. Regul Pept 1996; 67(1):33-7.
23. Smith-White M A, Potter E K. Structure-activity analysis of N-acetyl [Leu (28,31)] NPY 24-36: a potent neuropeptide Y Y(2) receptor agonist. Neuropeptides 1999 December; 33(6):526-33.
24. Doods H, Gaida W, Wieland H A, Dollinger H, Schnorrenberg G, Esser F, Engel W, Eberlein W, Rudolf K. BIIE0246: a selective and high affinity neuropeptide Y Y(2) receptor antagonist. Eur J. Pharmacol. 1999 November 19; 384(2-3):R3-5.
25. Pospisilik J A, Stafford S G, Demuth H U, Brownsey R, Parkhouse W, Finegood D T, McIntosh C H, Pederson R A. Long-term treatment with the dipeptidyl peptidase IV inhibitor P32/98 causes sustained improvements in glucose tolerance, insulin sensitivity, hyperinsulinemia, and beta-cell glucose responsiveness in VDF (fa/fa) Zucker rats. Diabetes 2002 April; 51(4):943-50.
26. Rahfeld J, Schierhorn M, Hartrodt B, Neubert K, Heins J. Are diprotin A (Ile-Pro-Ile) and diprotin B (Val-Pro-Leu) inhibitors or substrates of dipeptidyl peptidase IV? Biochim Biophys Acta 1991 January 29; 1076(2):314-6.
27. Ribozyme protocols: Turner, Philip C (editor). Humana Press, ISBN 0-89603-389-9, 512 pp. 1997.
28. Rossi J J. Ribozymes, genomics and therapeutics. Chem Biol 6, R33-7, 1999.
29. Ellington A D, Robertson M P, Bull J. Ribozymes in wonderland. Science 276, 546-7, 1997.
30. McManus M T and Sharp P A. Gene silencing in mammals by small interfering RNAs. Nat Rev Genet. 2002 October; 3(19):737-47.
31. Brody EN and Gold L. Aptamers as therapeutic and diagnostic agents. J Biotechnol 2000 March; 74(1):5-13.
32. Patel D J. Structural analysis of nucleic acid aptamers. Curr Opin Chem Biol 1997 June; 1(1):32-46.
33. Stull R A and Szoka F C Jr. Antigene, ribozyme and aptamer nucleic acid drugs: progress and prospects. Pharm Res 1995 April; 12(4):465-83.
34. Tajti J et al. The human superior cervical ganglion: neuropeptides and peptide receptors. Neuroscience Letters 263 (1999) 121-124.
35. Usman, N., Blatt, L. M. (2000) Nuclease-resistant synthetic ribozymes: developing a new class of therapeutics. J. Clin. Invest. 106:1197-1202.
36. Margaret F Taylor. Emerging antisense technologies for gene functionalization and drug discovery. DDT Vol. 6, No. 15 (Suppl), 2001.
37. Ray A, Norden, B. Peptide nucleic acid (PNA): its medical and biotechnical applications and promise for the future. FASEB J 14, 1041-1066, 2000.
38. Del Prete D, Anglani F, Ceol M, D'Angelo A, Forino M, Vianello D, Baggio B, Gambaro G. Molecular biology of diabetic glomerulosclerosis. Nephrol Dial Transplant. 1998; 13 Suppl 8:20-5. Review.
39. Satoh C, Satoh F, Takahashi K, Murakami O, Sone M, Totsune K, Yabe T, Ohneda M, Fukuda M, Sugimura K, Ogawa S, Nagakubo H, Sato T, Mouri T. Elevated plasma immunoreactive neuropeptide Y concentrations and its increased urinary excretion in patients with advanced diabetic nephropathy. Endocr J. 1999 February; 46(1):139-46.
40. Smith L E, Kopchick J J, Chen W, Knapp J, Kinose F, Daley D, Foley E, Smith R G, Schaeffer J M. Essential role of growth hormone in ischemia-induced retinal neovascularization. Science 1997 June 13; 276(5319):1706-9.
41. Smith L E, Shen W, Perruzzi C, Soker S, Kinose F, Xu X, Robinson G, Driver S, Bischoff J, Zhang B, Schaeffer J M, Senger D R. Regulation of vascular endothelial growth factor-dependent retinal neovascularization by insulin-like growth factor-1 receptor. Nat. Med. 1999 December; 5(12):1390-5.
42. Ozaki H, Seo M S, Ozaki K, Yamada H, Yamada E, Okamoto N, Hofmann F, Wood J M, Campochiaro P A. Blockade of vascular endothelial cell growth factor receptor signaling is sufficient to completely prevent retinal neovascularization. Am J Pathol 2000 February; 156(2):697-707.
43. Ruponen M, Rönkkö S, Honkakoski P, Pelkonen J, Tammi M, Urtti A (2001): Extracellular glycosaminoglycans modify cellular trafficking of lipoplexes and polyplexes. J Biol Chem 276, 33875-33880.

1. Method for treating or preventing a disease or disorder related to excessive formation of vascular tissue or blood vessels in a patient, wherein said disease or disorder is any form in which angiogenesis is involved, including neovascular glaucoma, any form of retinopathy, all proliferative retinopathies including proliferative diabetic retinopathy, retinopathy of prematurity, macular degeneration, maculopathy, micro- or macrovascular eye complications caused by diabetes, rubeosis iridis, or predisposition to vision loss and blindness, which are consequences of retinopathy,
said method comprising administering to said patient an agent affecting the NPY Y2 receptor, said agent being selected from the group consisting of
i) an NPY Y2 receptor antagonist,
ii) an NPY Y2 receptor antisense oligonucleotide complementary to any sequence of the human NPY Y2 receptor mRNA, said oligonucleotide having a length ranging from 7 to 40 nucleotides, or
iii) an agent being
an antibody raised against the Y2 receptor or raised against an Y2-specific epitope on the NPY peptide,
an aptamer affecting the Y2 receptor or a Y2-specific NPY-conformation,
a small interfering RNA molecule, or
a ribozyme, or
a peptide.

2. The method according to claim 1 wherein i) said agent also is a Y1-receptor agonist or antagonist, and/or ii) said agent also is a Y5-receptor agonist or antagonist.

3. The method according to claim 1 wherein the antisense oligonucleotide contains 15 to 25 nucleotides, wherein the antisense oligonucleotide optionally contains one or more chemical modifications of the nucleotides.

4. The method according to claim 3 wherein one or more of the internucleotide linkages are modified, and/or wherein the oligonucleotide contains locked nucleic acid (LNA) modifications and/or wherein the oligonucleotide contains peptide nucleic acid (PNA) modifications.

5. The method according to claim 3 wherein one or more of the sugar units are modified, and/or one or more of the internucleotide linkages are modified, and/or one or more of the bases are modified and/or the oligonucleotide is end-protected by an inverted deoxyabasic sugar.

6. The method according to claim 5 wherein some or all of the sugar units of the antisense oligonucleotide are 2′-deoxyribose and/or wherein the internucleotide phosphodiester linkages are replaced by phosphorothioate linkages.

7. The method according to claim 1 wherein the antisense oligonucleotide is selected from a group consisting of

| | |
|---|---|
| 5′- CCTCTGCACCTATTGGACCC -3′,; | (SEQ ID NO:2) |
| 5′- GTTTGTGGCCCGTATTGTTCC -3′,; | (SEQ ID NO:3) |
| 5′- GGCCACTGTTCTTTCTGACC-3′,; | (SEQ ID NO:4) |
| 5′- CTGCACCTATTGGACCCATT -3′ | (SEQ ID NO:7) |
| 5′- CTCTGCACCTATTGGACCCA -3′ | (SEQ ID NO:8) |
| 5′- GCCTCTGCACCTATTGGACC -3′ | (SEQ ID NO:9) |
| 5′- CAGCCTCTGCACCTATTGGA -3′ | (SEQ ID NO:10) |
| 5′- CGTATTGTTCCACCTTCATT -3′ | (SEQ ID NO:11) |
| 5′- CCGTATTGTTCCACCTTCAT -3′ | (SEQ ID NO:12) |
| 5′- CCCGTATTGTTCCACCTTCA -3′ | (SEQ ID NO:13) |
| 5′- GCCCGTATTGTTCCACCTTC -3′ | (SEQ ID NO:14) |
| 5′- GGCCCGTATTGTTCCACCTT -3′ | (SEQ ID NO:15) |
| 5′- TTTTCCACTCCCCCATTAAG -3′ | (SEQ ID NO:16) |
| 5′- ATTTTCCACTCCCCCATTAA -3′ | (SEQ ID NO:17) |
| 5′- CATTTTCCACTCCCCCATTA -3′ | (SEQ ID NO:18) |
| 5′- CCATTTTCCACTCCCCCATT -3′ | (SEQ ID NO:19) |
| 5′- CCCATTTTCCACTCCCCCAT -3′ | (SEQ ID NO:20) |
| 5′- CTCAATCAGCGAATACTCCC -3′ | (SEQ ID NO:21) |
| 5′- GATCTCAATCAGCGAATACT -3′ | (SEQ ID NO:22) |
| 5′- GCCACAATCTCAAAGTCCGG -3′ | (SEQ ID NO:23) |
| 5′- GGCCACAATCTCAAAGTCCG -3′ | (SEQ ID NO:24) |
| 5′- GCATTTTGGTGGTTTTTTGC -3′ | (SEQ ID NO:25) |
| 5′- CCAGCATTTTGGTGGTTTTT -3′ | (SEQ ID NO:26) |
| 5′- CCACACACACCAGCATTTTG -3′ | (SEQ ID NO:27) |
| 5′- CCACCACCACACACACCAGC -3′ | (SEQ ID NO:28) |
| 5′- CGCAAACACCACCACCACAC -3′ | (SEQ ID NO:29) |
| 5′- GCCAGCTGACCGCAAACACC -3′ | (SEQ ID NO:30) |
| 5′- GCCTTTCTGTAGTTGCTGTT -3′ | (SEQ ID NO:31) |
| 5′- GGAAAGCCTTTCTGTAGTTG -3′ | (SEQ ID NO:32) |
| 5′- GGCCGAGAGGAAAGCCTTTC -3′ | (SEQ ID NO:33) |
| 5′- CCACTGTTCTTTCTGACCTC -3′ | (SEQ ID NO:34) |
| 5′- GCCACTGTTCTTTCTGACCT -3′ | (SEQ ID NO:35) |
| 5′- GGGCCACTGTTCTTTCTGAC -3′ | (SEQ ID NO:36) |
| 5′- GGGGCCACTGTTCTTTCTGA -3′; | (SEQ ID NO:37) |

a combination of any of two or more of the aforementioned sequences or a combination of anyone of the aforementioned with another antisense oligonucleotide such as human vascular endothelial growth factor antisense VEGF-AS, 5′-GCCTCGGCTTGTCACAT CTGC-3′, (SEQ ID NO:41).

8. The method according to claim 7 wherein the sugar units of the antisense oligonucleotides are 2′-deoxyribose and wherein the internucleotide linkages are phosphorothioate linkages.

9. The method according to claim 1 wherein said agent is a combination of agents having ability to affect the action of NPY Y2 receptor.
